(19) United States
(12) Patent Application Publication
(10) Pub. No.: US 2007/0239260 A1
(43) Pub. Date: Oct. 11, 2007

(54) Devices and methods for tissue welding

(76) Inventors: Daniel V. Palanker, Sunnyvale, CA (US); Alexander B. Vankov, Menlo Park, CA (US)

Correspondence Address: MORRISON & FOERSTER LLP, 755 PAGE MILL RD, PALO ALTO, CA, 94304-1018, US

(21) Appl. No.: 11/731,644

(22) Filed: Mar. 29, 2007

Related U.S. Application Data
(60) Provisional application No. 60/787,783, filed on Mar. 31, 2006.

(51) Int. Cl. A61F 2/06

(52) U.S. Cl. 623/1.15

(57) ABSTRACT

Tissue implants configured to adhere to biological tissues when activated by electrical energy may include an electrically conductive structure, a connector releasably connected to the electrically conductive structure, and a thermally crosslinkable coating covering at least the exposed portion of the electrically conductive structure. These tissue implants may be used for welding tissues to other tissues, or for welding tissue to the implant, and thus may be used to attach implants within a body, or for therapeutic uses. These implants may be used for wound closure or to create occlusions. Thermal damage to the tissue may be minimized by use of the thermally-crosslinkable material having a resistivity higher than that of the adjacent tissue.

### CROSS-REFERENCE TO RELATED APPLICATIONS

This application claims priority to U.S. Provisional Patent Application Ser. No. 60/787,783, filed Mar. 31, 2006, which is herein incorporated by reference in its entirety.

### TECHNICAL FIELD

This application generally relates to the field of surgery. In particular, the application relates to the electrosurgical adhesion of biological tissue to an implant and/or to tissue.

### BACKGROUND

There are many ways to adhere tissue to itself or to an implantable device or surface, including tissue adhesives (e.g., “glues”), laser tissue welding, and electrical tissue welding. Examples of these methods may be found, for example in U.S. Pat. No. 6,562,037, U.S. Pat. No. 6,669,694, U.S. Pat. No. 6,733,498, and U.S. Pat. No. 5,749,895. However, such methods include sufficient drawbacks preventing their widespread use and adoption. This is particularly unfortunate, because tissue adhesion (e.g., adhering materials to tissue or tissue to other tissue) holds the promise of profound medical benefit in areas such as wound closure and healing, implantation of medical devices, and surgical interventions.

For example, common techniques for closing an incision include suturing, clamping, stapling and gluing. These techniques have a number of well-known disadvantages such as: trauma to adjacent tissues, leaving, pinching or compressing tissue (delaying healing and/or causing inflammation), allergic reaction, complexity of use, and the need for expensive equipment. For example, typically “tissue glues” (e.g., cyanoacrylates), are difficult to apply, may release harmful by-products when curing, may heat when curing, and may themselves induce an immunogenic response in the patent. Further, they may prevent accurate and rapid adhesion of the tissue.

More recently, techniques such as laser radiation, heated tools and the passing of high frequency current directly through the parts of tissue have been suggested as alternative means of adhering tissue. Most of these methods rely upon tissue protein denaturation caused by heating to affect tissue adhesion. When the temperature of most tissues exceeds 55° C., the tissue proteins denature and coagulate. Thus, if two edges of tissue are connected and heated, the entanglement of molecules (e.g., collagen, albumen, etc.) may result in their bonding. For example, see: “Experimental study on the healing process following laser welding of the cornea,” Journal of Biomedical Optics, March/April 2005, v. 10, no. 2, p. 1-7; “Diode laser welding for cornea suturing: an experimental study of the repair process,” Proceedings of the SPIE—The International Society for Optical Engineering (2004), vol. 5314, no. 1, p. 245-52; “Conference: Ophthalmic Technologies XIV,” 24-27 Jan. 2004, San Jose, Calif., USA; “An experimental study on laser-induced suturing of venous grafts in cerebral revascularization surgery Progress in Biomedical Optics and Imaging,” Proceedings of SPIE, 2005, v. 5686, p. 276-281. Additional proteins may be added between the edges of the tissue, so that upon heating, these additional proteins denature, and may straighten and entangle. If two edges of tissue contact each other and are heated, the entanglement of protein molecules may result in their bonding. Typically, the higher the temperature, the faster and better is the coagulation. However, at temperatures exceeding 100° C., the tissue becomes dehydrated, and its electric resistance increases, which leads to further temperature rise and charring of the tissue. Further, this heating of the tissue is relatively non-specific and results in undesirable damage to the tissue (or adjacent tissues), as well as deformation (e.g., shrinking), scarring, and other undesirable consequences.

Recently, laser tissue welding has been proposed as an alternative means for tissue adhesion. Research efforts have investigated the laser techniques in blood vessel surgery. This technique has not been widely accepted for general clinical use, however, because of the technical complexity of its implementation and because of inadequate surface energy release. Typically in laser tissue welding, the tissue (e.g., a wound) is filled with a dye solution that will be illuminated by the laser. The dye absorbs the laser radiation, heating the nearby tissue and allowing the collagen to denature, as described above. Thus, laser tissue welding requires that the dye (and/or tissue) receive adequate light energy to heat the adjacent tissue. The wavelength of the laser must have a sufficiently long penetration depth to reach the necessary regions, while minimizing burns on the sides of the wound. In practice, laser tissue adhesion does not uniformly reach all regions, because it is limited by the application of light, since tissue scattering of light and absorption by the dye results in deeper tissue layers receiving less laser energy than superficial layers. Furthermore, the use of light to cause tissue adhesion may be time-intensive, since light must typically be applied from different locations (e.g., at different angles). FIGS. 7A and 7B show examples of laser tissue welding.

In FIG. 7A, the tissue 709 includes a tear or cut 707, into which a light-activatable sealant 705 has been added so that it can be sealed to close the cut 707. A light source 701 is used to apply light to activate the sealant 705. The sealant 705 is typically a light absorber. Because the light does not have uniform access to the entire cut region, the absorber or sealant does not uniformly absorb light, and may therefore not completely seal or weld the cut 707. This is particularly true in other patterns in which sealant is applied, including circular patterns (e.g., around lumen), as shown in FIG. 7B. In FIG. 7B the upper portion of the circular cross-section 717 is heated (by absorption of light from the absorber 705) more than the lower region of the circular cross-section 717.

Existing electrosurgical methods for welding or adhering to tissue also suffer from numerous drawbacks. Typically, electrical devices (e.g., bipolar or monopolar electrosurgical devices) apply energy to tissue to heat the tissue electrically. When connecting tissue to tissue (e.g., sealing wounds or closing blood vessels), pressure is typically applied so that as the collagen in the tissue is denatured and renatured, it adequately combines with the overlapping tissue. Such electrosurgical treatment of tissue has proven difficult to use, and often results in therapeutically undesirable consequences such as burning, tissue necrosis, and non-uniform adhesion. Specifically, it has been notoriously difficult to correctly determine and use electrical signal parameters to achieve adhesion of tissue. This is due, at least in part, to the fact that tissue has an electrical resistance which can vary widely depending on many factors such as tissue structure and thickness as well as the tool/tissue contact area which difficult to reliably control. If too little current is applied, then any resulting tissue joint can be spongy, weak and unreliable. On the other hand, if too much current is applied, then the working surface of the electrode can stick to the tissue so that removal of the electrode causes bleeding and possible injury. Also, the tissue in the overly-heated zone can become desiccated and charred. Therefore, high frequency coagulative devices have been limited mostly to use for hemostasis of blood vessels of relatively small diameter.

Thus, there is a need for reliable tissue adhesion methods, devices and systems that avoid many of the problems described above. Described herein are methods, devices and system that may address the problems raised above.

### SUMMARY OF THE INVENTION

The devices, systems, and methods described herein illustrate devices, methods and systems for adhesion or welding of tissue. In particular, the implants described herein may be electrically activated to adhere to tissue by activating a thermally crosslinkable material that is in contact with an electrically conductive structure. At least a subset of the implants described herein may also be referred to as electric tissue weld devices. Implant may also be referred to as adhesive implants. For example, an implant may be coated with a thermally crosslinkable material. Coated implants may be inserted into tissue and activated to adhere to tissue. Activation of the implants described herein results in a localized temperature rise that thermally activates the crosslinkable material (e.g., albumin), while avoiding overheating adjacent tissue.

For example, an implant (e.g., an adhesive implant or an electric tissue weld device) as described herein may be configured to adhere to a biological tissue when activated by electrical energy. An implant may include an electrically conductive structure, a connector releasably connected to the electrically conductive structure, and a thermally crosslinkable material in contact with the electrically conductive structure, such as a thermally crosslinkable coating that covers at least a portion of the electrically conductive structure. The electrically conductive structure may be configured to have one or more tissue-facing surfaces. The tissue-facing surfaces typically contact the thermally crosslinkable material so that they are in electrical or thermal contact with the thermally crosslinkable material, and the thermally crosslinkable material is in turn in electrical contact with the tissue

In some variations, the electrically conductive structure is configured so that it may be left (e.g., implanted) in a subject's tissue after activation of the adhesive (thermally crosslinkable) coating. Thus, the electrically conductive structure may be configured for implantation into the tissue. In some variations, the implant (and particularly the electrically conductive structure of the implant) is configured so that it is degraded upon electrical activation (e.g., during crosslinking of the thermally crosslinkable material). The electrically conductive structure may be configured as a pad, a frame, a stent, a foil, a mesh, or the like. In some variations, the electrically conductive structure is only a portion of the implant. Thus, any appropriate medical implant may include a portion that is electrically conductive. The electrically conductive portion typically extends to an external or tissue-facing surface (e.g., the surface to contact and eventually adhere to the tissue) of the implant. The electrically conductive structure may be made of any appropriate material. For example, the electrically conductive structure may be made at least partially of an electrically conductive polymer, or of an electrically conductive metal such as titanium, gold, nickel, implant-grade stainless steel, cobalt alloys, platinum, or alloys or combinations of these. All or part of an implant (including the electrically conductive structure) may be made of a bioabsorbable material. As described herein, the implant can be made of any biocompatible or bioabsorbable material, and may include implants comprising a metal coating (e.g., a metal coating over an absorbable material) that can be configured for to be destroyed during or by activation. For example, the application of electrical energy to the electrically conductive region may cause the breakdown of the electrically conductive region.

As mentioned above, the electrically conductive material of the implant may be degraded (e.g., broken down, dissolved, etc.) by electrical activation of the implant. Electrical activation to dissolve the implant may be the same activation (e.g., for the same duration and amount) that is used to perform the tissue welding or crosslinking of the thermally crosslinkable material. In some variations, complete (or partial) removal or degradation of the electrically conductive material is achieved by application of additional electrical energy after welding. Thus, after insertion of the implant and activation of the thermally crosslinkable material, the electrically conductive material may be dissolved, effectively removing it from the tissue (or allowing relatively quick removal by the tissue).

The implant may also include a connector that is releasably connected to the electrically conductive structure. The connector is typically configured to connect the implant to an energy source (e.g., power supply) that can apply electrically energy to activate the thermally crosslinkable coating by applying current through the electrically conductive structure. In general, the connector is detachable from the implant, and can be removed after (or during) activation to leave the implant within the tissue. For example, the connector may be releasably connected to the implant by a frangible connection. In some variations, the connector is releasably connected by an electrically erodible connection. As mentioned, in some variations, the electrically conductive material of the implant erodes, disconnecting from the connector (another example of an erodible or frangible connector). Any appropriate connector may be used. For example, the connector may be a plug, a clamp, etc. One example of a connector comprises a penetrating electrode. The implant (e.g., the thermally crosslinkable material of the implant) may be activated by contacting this electrode to a voltage/current source. If a small gap is present between a part of the electrode (e.g., the tip) and the conductive region of the implant, a high activation voltage will cause breakdown in this gap, and establish an electric contact. Thus, the connector does not need to be in fixed connection with the electrically conductive region.

The thermally crosslinkable material is typically a material that is thermally polymerizable, and may be manufactured to have a resistivity that is higher than that of at least some biological tissue (e.g., the tissue into which it is implanted). The higher resistivity of the thermally crosslinkable material may result in the material being preferentially heated by electrical activation. In one example, the thermally crosslinkable coating comprises albumin. The resistivity of the albumin may be adjusted by adjusting its ion concentration (or by adjusting the ion concentration of the surrounding tissue. For example, the coating of albumin may be treated to remove ions.

As mentioned, the thermally crosslinkable coating typically has a resistivity that is higher than biological tissue. In some variations, the thermally crosslinkable coating has a resistivity higher than 100 Ohm*cm. The resistivity of the thermally crosslinkable coating may also increase during thermal crosslinking. The resistivity of the thermally crosslinkable coating may also increase by vaporization during heating.

Other examples of thermally crosslinkable coatings include collagens, fibrins, and some polysaccharides. As described in greater detail below, coatings of these materials may be thermally polymerized, and may have resistivities that are greater than tissue. More than one electrically conductive coating may be used.

In variations of the device including coating of thermally crosslinkable material, any appropriate thickness of thermally crosslinkable coating may be used. For example, the coating may be greater than 10 μm thick. The coating of thermally crosslinkable material may cover the entire exposed (e.g., outer) surface of the electrically conductive structure, preventing “short circuiting” of the implant's electrically conductive structure where it may contact tissue having a lower resistivity. Portions of the electrically conductive structure that are not coated with thermally crosslinkable coating may be insulated, or otherwise protected from contacting the tissue. A coating generally refers to a portion or region of material (e.g., thermally cross-linkable material) that is in contact with a surface of the electrically conductive structure and is exposed to tissue when implanted into the tissue. Thus a coating may be a layer, a region, or the like.

One variation of an electrically activated adhesive implant is configured to adhere to a biological tissue when activated by electrical energy. This implant includes an electrically conductive structure having a connector configured to connect the electrically conductive structure to a power supply. At least a portion of this electrically conductive structure is coated with a thermally crosslinkable coating having a resistivity higher than that of the biological tissue.

Another example of an electrically activated adhesive implant that is configured to adhere to a biological tissue when activated by electrical energy includes an electrically conductive structure having a connector configured to connect the electrically conductive structure to a power supply. At least a portion of the electrically conductive structure is coated with a thermally crosslinkable coating, wherein the resistivity of the thermally crosslinkable coating becomes higher than that of the biological tissue during thermal cross-linking of the coating to the biological tissue.

In some variations, the adhesive implant is configured as an electric tissue weld device, an electric bandage, or an electric glue that may be inserted against or into the tissue (e.g., between the sides of a tissue wound) to secure tissue. For example, an electric tissue weld device may hold (or even seal) the tissue together.

Also described herein are methods of attaching an implant to a biological tissue. In one variation, the method includes the steps of inserting an implant into the tissue and activating a thermally crosslinkable material applied to the tissue an in electrical contact with the implant. For example, the thermally crosslinkable material may be present as a coating on the implant. The implant may include any of the implants described herein, including an implant having an electrically conductive structure with a connector that is configured to connect the electrically conductive structure to a power supply, wherein at least a portion of the electrically conductive structure is coated with a thermally crosslinkable coating. The resistivity of the thermally crosslinkable coating typically becoming higher than that of the tissue upon heating and cross-linking of the thermally crosslinkable coating.

In some variations, the method of attaching an implant to a biological tissue also includes the step of disconnecting the electrically conductive structure from the power supply. The step of activating the thermally crosslinkable coating may include applying electrical energy to the electrically conductive structure.

Also described herein is a method of welding a biological tissue. As used herein the term “tissue welding” may mean adhesion of a tissue to another tissue (or another region of tissue) and/or to an implant. This method of tissue welding typically includes the steps of placing an implant adjacent to the tissue (wherein the implant comprises an electrically conductive structure releasably connected to a power supply and a thermally crosslinkable coating covering at least a portion of the electrically conductive structure), and applying electrical energy to the electrically conductive structure of the implant to at least partially crosslink the thermally crosslinkable coating of the implant with the tissue. In some variations, the step of applying electrical energy is an activating step wherein the thermally crosslinkable material is activated to cause one or more regions of tissue to crosslink to the implant, thus connecting the tissue and the implant. This method may be used in any appropriate application, including tissue closure (e.g., wound healing, etc.), device anchoring, vaso-occlusion, etc.

In one method for welding a biological tissue, electrical energy is applied to an implant that is in contact with a thermally crosslinkable material, raising the temperature of the thermally crosslinkable material, causing the crosslinkable material to adhere (or stick) to the tissue. For example, the crosslinkable material may form crosslinks (e.g., covalent bonds) with proteins or other crosslinkable materials in the tissue. In some variations, the thermally crosslinkable (or polymerizable) material is applied to the tissue (e.g., by coating, spraying, painting, pouring, dipping, etc.) and then an implant having an electrically conductive region is placed in electrical contact with the thermally crosslinkable material. Electrical energy applied to the implant then activates the thermally crosslinkable material. In some variations, the thermally crosslinkable material is applied to the implant and the implant is then applied to the tissue. In some variations, additional thermally crosslinkable material that is not coated to the implant is added to the tissue either before, during, or after insertion of the coated implant. In any of the methods described herein, more than one type or class of thermally crosslinkable material may be used. For example, thermally crosslinkable materials having different electrical conductivities may be used (e.g., in different regions or layers of the implant).

It is notoriously difficult to cut some tissues (e.g., vascularized organs such as liver) due to massive bleeding from the dissected blood vessels at the edges of the cut. To stop bleeding, deep coagulation is often required; however this coagulation may damage a significant part of the tissue. A thermally crosslinkable material (such as albumin or fibrin etc.) may be applied so that, upon heating, it will seal the bleeding blood vessels, without requiring the need for deep coagulation. Methods and devices for preventing bleeding using a thermally polymerizable material are also described herein.

The details of one or more embodiments of the tissue adhesion methods, systems and devices (e.g., implants) are set forth in the accompanying drawing and in the description below. Other features, objects, and advantages of the invention will become apparent from the description and drawings, and from the claims.

### BRIEF DESCRIPTION OF THE DRAWINGS

The accompanying drawings, which are incorporated in and form a part of this specification, illustrate embodiments of the invention and, together with the description, serve to explain the principles of the invention:

FIGS. 1A-1D illustrates the effect of heat deposition power as a function of the resistivity of a thermally crosslinkable material and the resisitvity of the tissue.

FIGS. 2A-2E are example of electrical tissue weld devices as described herein.

FIGS. 3A and 3B illustrate electrical tissue welding as described.

FIG. 4 is one example of an electrical tissue welding implant configured as a stent, as described herein.

FIG. 5 shows a histological section though a region of smooth cardiac muscle to which an implant including a thermally crosslinkable material has been applied.

FIG. 6 is a load curve for an implant applied to cardiac endothelium.

FIGS. 7A and 7B illustrate two prior art laser tissue welding methods.

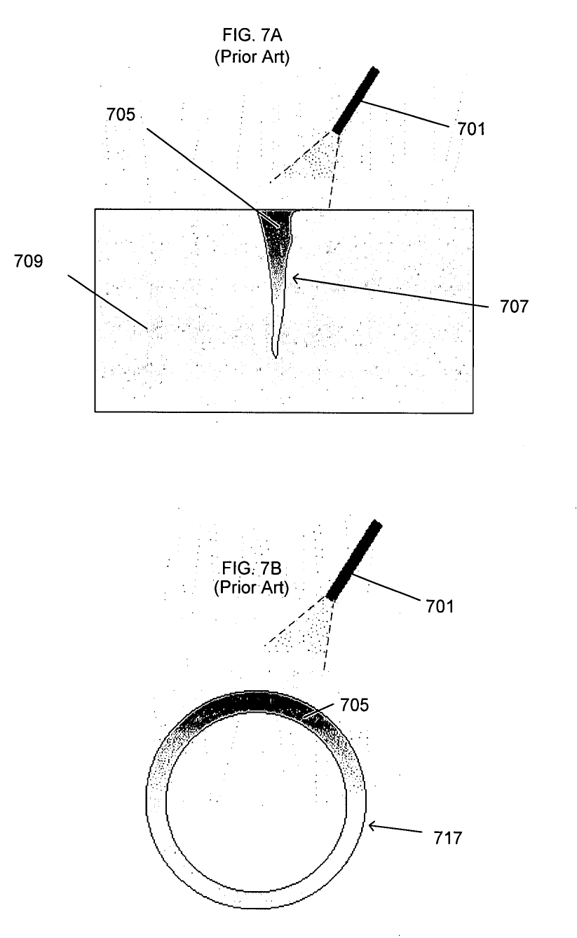

### DESCRIPTION OF INVENTION

Described herein are devices, methods and systems for electrical welding of biological tissue using a thermally crosslinkable (or polymerizable) material. The devices described herein are exemplified by implants that may be inserted into a biological tissue to adhere to the tissue. Implants may be configured as medical devices (e.g., medical implants such as stents, catheters, pacemakers, biosensors, etc.). In some variations, implants are configured for wound closure.

In general, the implants are configured to adhere to a biological tissue when activated by electrical energy. The implant typically includes one or more electrically conductive structures, and a connector (or connectors) that are releasably connected to the electrically conductive structure. The electrically conductive structure may be used in conjunction with a thermally crosslinkable coating (e.g., a tissue “solder”). In particular, the implant may be coated with a thermally conductive material. Although it may be referred to as a ‘coating,’ a thermally conducive material may be applied or attached to the implant by any appropriate manner, so long as it is in contact with the electrically conductive structure of the implant. For example, the thermally crosslinkable material may be layered over the electrically conductive region.

In operation, the application of electrical energy to an electrically conductive structure of the implant heats the thermally crosslinkable material, causing the material to polymerize, and adhere to the tissue. Further, because the resistivity of the thermally crosslinkable material is typically much higher than the relative resistivity of the tissue, the applied electrical current raises the temperature of the thermally crosslinkable material substantially more than the surrounding tissue, preventing or minimizing thermal damage to the tissue, while activating the adhesive properties of the thermally crosslinkable material that contacts the implant.

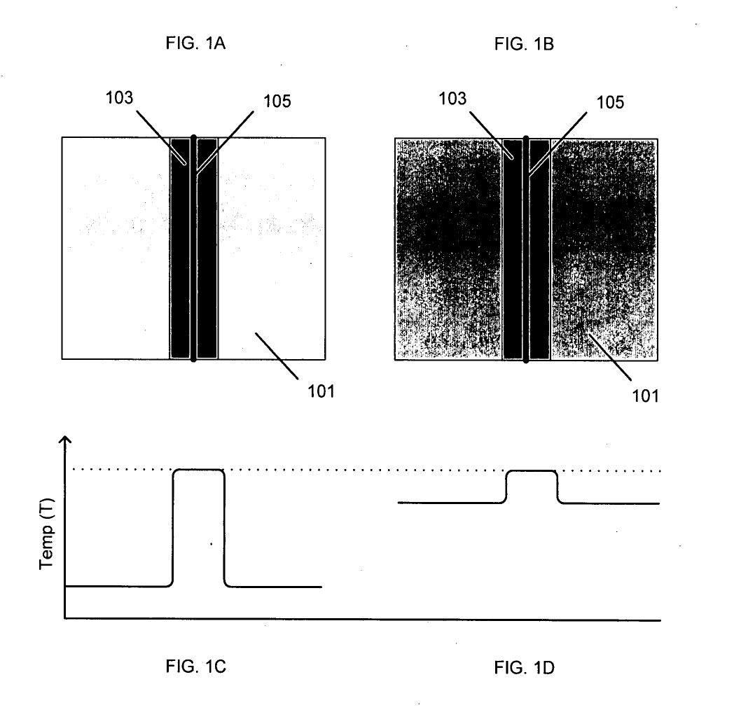

FIGS. 1A-1D illustrate the theory behind this specificity. The local temperature rise necessary for activating the thermally crosslinkable material originates from the current applied. When the uninsulated portions of the electrically conductive member is surrounded by a thermally crosslinkable material, current must pass through this material before entering the tissue and passing to ground (e.g., a ground electrode). The Joule heat deposition of power resulting from the application of current may be calculated by the relationship:
W=σ*j² [1]
where j is current density, and σ is the resistivity of the material.

Thus, when any particular current density is applied, the amount of heat resulting will be proportional to the resistivity of the material. In FIGS. 1A and 1B, this is illustrated in the context of the localization of heat sufficient to polymerize (or crosslink) the thermally crosslinkable material coated on an implant. FIGS. 1A and 1B show a thermally crosslinkable material 103 that is in contact with an electrical conductor 105, which may be part of an implant, as described herein. The electrical conductor 105 (e.g., a metal foil or mesh) is placed between two tissue regions 101. In FIG. 1A, the tissue has a resistivity, σₜᵢₛₛᵤₑ, that is much less than the resistivity of the thermally crosslinkable material (solder), σ_{solder}. In this example, the thermally crosslinkable material is albumen. Thus, in FIG. 1A, σₜᵢₛₛᵤₑ<<σ_{solder}. The application of energy through the electrically conductive structure 105 that is surrounded by the thermally crosslinkable material 103 when implanted into the tissue results in a steep difference in the Joule heat deposition between the thermally crosslinkable material 103 and the surrounding tissue 101. Thus, the change in temperature of tissue (ΔTₜᵢₛₛᵤₑ) is much less than the change in temperature of the thermally crosslinkable material (ΔT_{solder}). FIG. 1C shows this difference in temperature qualitatively across the tissue and implant sections (along the x-axis). The magnitude of temperature is shown in the vertical axis. Thus, in the situation shown in FIG. 1A, when the σₜᵢₛₛᵤₑ<<σ_{solder}, the change in temperature of the tissue 101 is much less than the change in temperature of the thermally crosslinkable material 103 upon application of current to the electrical conductor 105, as shown in FIG. 1C. In general, the change in temperature (ΔT) may be related to the Joule heat deposition power by the relationship:

$$Δ T = \frac{{j}^{2} * σ * t}{mC} \qquad [ 2 ]$$

This effect may be extremely beneficial to the implant, because when the resistivity of the thermally crosslinkable material is much higher than that of the surrounding tissue, the high temperature necessary to polymerize the thermally crosslinkable material does not spread deeply into the tissue, preventing excessive thermal damage to the surrounding tissue. Furthermore, crosslinking the thermally crosslinkable material causes the material to adhere to the tissue, even after it has cooled down, and in some variations the further crosslinking of the thermally crosslinkable material increases the resistivity. Thus, thermal damage to the tissue may be minimized because of the higher resistivity of the thermally-crosslinkable material than that of the adjacent tissue. Even in the σ_{solder}>>σₜᵢₛₛᵤₑ scenario shown in FIGS. 1A and 1C, the temperature of the surrounding tissue 101 (in particular, the temperature of the tissue immediately adjacent to the thermally crosslinkable material) may increase in temperature, which may enhance crosslinking of the tissue to the thermally crosslinkable material. In this example, the temperature of the adjacent tissue may be raised only locally (e.g., close to or in contact with) the thermally crosslinkable material, preventing thermal damage to the larger tissue area.

In contrast, when the resistivity of the thermally crosslinkable material (σ_{solder}) is approximately equivalent (or near) the resistivity of the tissue into which the implant is being inserted (σₜᵢₛₛᵤₑ), the Joule heat deposition is approximately equivalent, resulting in heating of the surrounding tissue. This is illustrated in FIG. 1B, and the resulting temperature profile is shown in FIG. 1D. As shown in FIG. 1D, the change in temperature of the tissue (ΔTₜᵢₛₛᵤₑ) is approximately the same as the change in temperature of the thermally crosslinkable material (ΔT_{solder}), when, as in FIG. 1B, the σ_{solder}≈σₜᵢₛₛᵤₑ. In this case, the heating may cause thermal damage, shrinkage, and may otherwise damage the surrounding tissue, particularly when the temperature for adhesive crosslinking is relatively high.

The implants described herein may take advantage of the relationship described above. For example, the implants may include a thermally crosslinkable material that is in electrical contact with an electrically conductive structure so that current can be applied through the thermally crosslinkable material. Any of the devices or system described herein may also include a return or ground electrode. The resistivity of the thermally crosslinkable material maybe be selected so that it is significantly higher than the resistivity of the tissue into which the device will be implanted.

Tissue resistivity has been studied, and experimental and theoretical models of tissue resistivity are well known. Examples of estimates of tissue resistivity in different tissues include: blood (1.5 Ohms*m), Liver (3.5 Ohms*m), fat (20.6 Ohms*m), bone (16.6 Ohms*m), lung (7-23 Ohms*m), etc. Exemplary lists of tissue resistivities are provided in Geddes and Baker (Geddes L A, Baker L E, “The specific resistance of biological material—A compendium of data for the biomedical engineering and physiologist.” Med Biol. Eng. 5: 271-93, 1967), Barber and Brown (Barber D C, Brown B H, “Applied potential tomography.” J. Phys. E.: Sci. Instrum. 17: 723-33; 1984), and Stuchly and Stuchly (Stuchly M A, Stuchly S S, “Electrical properties of biological substance.” In Biological Effects and Medical Applications of Electromagnetic Fields, ed. O P Gandhi, Academic Press, New York, 1984).

Thus, in some variations, the resistivity of the thermally crosslinkable material is greater the resistivity of the tissue into which the material (or an implant including the material) is to be applied. For example, the thermal resistivity of the thermally crosslinkable material may be greater than an average or approximate thermal resistivity of human tissue, and particularly of soft tissues such as skin, muscle, etc. The thermal resistivity of the tissue may be measured for an individual, or it may be estimated from population data.

As described in greater detail below, the resistivity of a thermally crosslinkable material may be adjusted. For example, the resistivity may be adjusted by modifying the ion concentration of the crosslinkable material, and by otherwise modifying the composition of the thermally crosslinkable material. Electrical current typically passes from the electrically conductive region of the implant and through the thermally crosslinkable material on the way to ground. In general, the electrically conductive structure of an implant may be surrounded (e.g., by coating, etc.) with an adequately thick layer of thermally crosslinkable material so that the electrically conductive structure does not contact the tissue directly (e.g., so that it does not contact a lower resisitvity material), which may change the current path, and alter the heating of the thermally conductive layer. Thus, an electrically conductive region, structure or layer of an implant may be insulated in regions where the thermally conductive layer does not cover the electrically conductive region. This electrical insulation may prevent current from passing into the tissue without first passing through the thermally crosslinkable material.

The implant may include any appropriate electrically conductive structure. The electrically conductive structure may make up the majority of the implant structure, or just a portion of the implant structure. The electrically conductive structure is generally an electrode having one or more surfaces that may be in contact with the thermally conductive material. These surfaces may be electrically conductive surfaces.

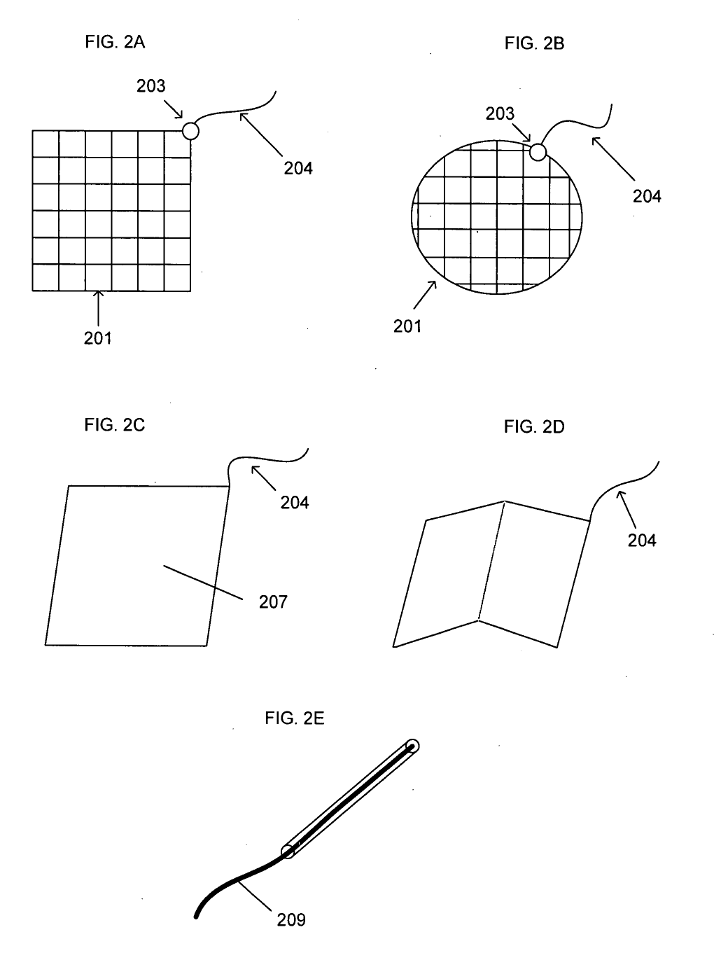

The implants described herein may be configured for wound closure. For example, the implant may be a foil, mesh, or pad having an exposed surface that is coated with a thermally crosslinkable material. One variation of an implant for wound closure is an electric tissue weld device. An electric tissue weld device may include an electrically conductive structure that is coated with a thermally crosslinkable material. FIGS. 2A-2E illustrate different examples of electric tissue weld devices. For example, in FIG. 2A, the electric tissue weld device (or implant) includes an electrically conductive structure configured as a mesh 201. The mesh is coated with a thermally crosslinkable material (e.g., albumen), and is connected via a connector, which includes an electrical attachment 203 and a connecting cable 204, to a power source for applying electrical energy to the implant. A similar implant is shown in FIG. 2B, wherein the electrically conductive structure is configured as a circular grid. As already described, the electrically conductive structure may be any appropriate shape or configuration that allows current to from the electrically conductive structure and through the thermally crosslinkable material that surrounds the un-insulated electrically conductive structure.

FIG. 2C is another variation of an electric tissue weld device in which the electrically conductive structure is configured as a foil 207. The foil is also connected (or connectable) to a power supply via a connector including a connecting cable 204 and an electrical attachment 203. A separate electrical attachment is not shown in FIG. 2C, however one may be optionally used. In some variations the electric tissue weld devices may be flexible or conformable so that they may be bent to fit in, over or across a wound or cut within the tissue. Thus, the electric tissue weld device may be made of a ductile material, and/or a flexible material. For example, any of the mesh-type electric tissue weld devices as shown in FIGS. 2A and 2B may be bent either before, during, or after insertion into the tissue. FIG. 2D shows the implant of FIG. 2C after it has been bent along one axis.

The connectors, and particularly the connecting cables 204 shown in FIGS. 2A-2E are insulated along their length (e.g., towards their proximal ends). In some variation the connectors are at least partially coated with a thermally crosslinkable material.

Any of the devices (including the electric tissue weld devices) may be a one-dimensional (e.g., liner) implant device, a two-dimensional (e.g., planar) implant device, or a three-dimensional implant device. FIGS. 2A-2D illustrate planar implant devices in which the electrically conductive structure forms a plane. FIG. 2E is a linear electric tissue weld device configured as a wire. The distal end of the linear electric tissue weld device 209 is coated with a thermally crosslinkable material, and the proximal end is an insulated wire.

The electrically conductive region may comprise any appropriate electrically conductive material. In particular, the electrically conductive region may comprise a biocompatible material. Example of electrically conductive materials may include metals such as titanium, gold, platinum, nickel, implant-grade stainless steel, cobalt alloys, etc. In some variations, the electrically conductive structures may provide structural support to the implant as well as conducting electrical energy to activate the thermally crosslinkable material.

The electrically conductive structure may be configured as an electrode. For example, the electrically conductive material may include a flattened contact tissue-facing surface. This tissue-facing surface may be configured to conform to the tissue (e.g., it may be planar, or curved so that it is complementary to the tissue surface(s) that it will contact. As mentioned above, the implant may be flexible or shapeable. For example, the implant may comprise a mesh that may be bent or shaped to best fit within the tissue.

In some variations, the implant may include an electrically conductive structure on multiple sides. For example, a pad or foil useful in wound closure may have external electrically conductive surfaces (e.g., electrodes) coated with thermally crosslinkable material on either side, so that it can be adhered to both sides of a tissue opening. FIGS. 2A-2D show examples of such implants.

The implant may be configured as a patch or foil that can be used to close a wound. The majority of the outer surface of the implant may be an exposed portion of the electrically conductive material that is coated with a thermally crosslinkable material. For example, the implant may comprise a frame that is coated with thermally crosslinkable material. The frame may be implanted into a subject's body and placed in contact with one or more tissues (e.g., to close a wound, or to graft tissues together). Once the implant contacts the tissues, the electrically conductive structure is activated, crosslinking it to the tissues that contact it, and effectively gluing the implant in position. Thus, the electrically conductive region and the associated thermally crosslinkable material may form a tissue weld that is controllably activated by the application of electrical energy. Before the activation of electrical energy, the implant may not appreciably adhere to the tissue.

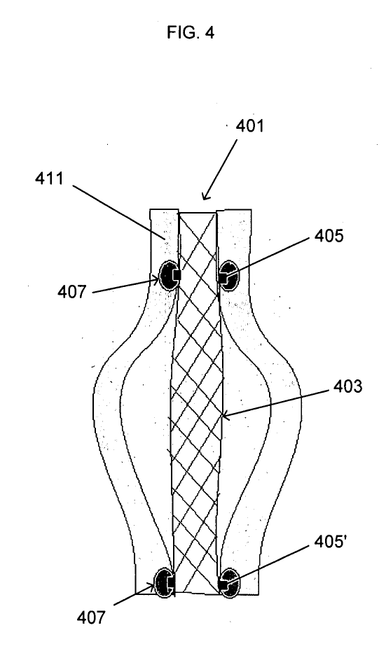

In some variations, the electrically conductive structure is included as part of an implant which has additional features, such as structural support, drug delivery features, sensors, stimulators, or the like. For example, the implant may be configured as a stent. FIG. 4 illustrates an implant configured as a stent 401 for insertion into an aorta. In FIG. 4, the stent comprises a support body 403 and electrically conductive structures 405 that are located on outer surfaces (e.g., tissue-facing surfaces) of the stent along the length of the stent. These electrically conductive structures 405 are shown in cross-section near the ends of the stent in FIG. 4. The electrically conductive structure 405 may be a ring or may be discrete electrodes. At least the outward-facing surface of the exposed electrically conductive structure is coated with a thermally crosslinkable material 407, so that when the electrically conductive structures 405 are activated by the application of electrical energy, the thermally crosslinkable material preferentially heats and polymerizes, adhering the stent to the surrounding tissue. In the example of FIG. 4, the implant (configured as a stent) is fixed within an aortic aneurysm. Thus, activation of the stent by applying energy to the electrically conductive structure(s) of the stent causes it to adhere to the wall of the aorta 411, as shown in FIG. 4. Once the implant is activated and the thermally crosslinkable material has been crosslinked, the implant cannot be easily removed.

Any appropriate connector (e.g., electrical connector) may be used to apply electrical energy to the electrically conductive region. In particular, the electrically conductive member may be connected via a removable or releasable connector. Since the implants are typically retained by the body after application of electrical energy to activate the thermally crosslinkable material (or the implant may partially disintegrate upon activation), the electrically conductive structure of the implant should be separable from the electrical power supply.

In some variations, the connector includes either an electrical attachment or an electrical attachment and a connecting cable. The electrical attachment connects the electrically conductive structure of the implant to a power supply. In some variations, the connector also includes a connecting cable that connects the electrically conductive structure to the power supply. For example, the connector may include an electrical attachment that is configured as a plug or dock for a lead from a power supply. The lead may be a connecting cable that is attached (or attachable) to the electrically conductive region of the implant and to a power supply. Thus, the implant may comprise a connector configured as a plug or other attachment. In some variations, the implant includes a mate for a lead from a power supply. In some variations, the connector may be a pad or contact surface against which a lead, probe, clip, wire, etc. may make an electrical contact with the implant.

An implant may also include a frangible connector for connection to the power supply. A frangible connector may be removed, broken or dissolved after the implant has been attached by the application of electrical energy. Completely removing the connector may make the implant smaller, which may be advantageous. For example, the implant may include a thin wire through which electrical energy is applied to the electrically conductive region. Examples of this are shown in the implants of FIGS. 2A-2E.

In some variation, the connector for connection to the power supply comprises a contact point or region for contacting the electrically conductive portion of the implant with a connector to a power source (e.g., a wire, cable, etc.). Thus, the implant can be activated by touching the electrically conductive region with a wire connected to the power supply. The wire connected to the power supply may be insulated everywhere except for the point of contact with the conductive structure in the implant. In some variations the point of contact is insulated by the insulation is removable (e.g., pierceable) by a connector that may connect to the power supply. After activation of the thermally crosslinkable material by applying power from the power supply, the wire is then removed from the contact point, leaving the attached implant in place.

It may also be possible to apply energy to activate the thermally crosslinkable material in contact with an electrically conductive member without using a connector. For example, an external electromagnetic field may be applied. An external electromagnetic field may induce a current that heats the thermally crosslinkable material. In one variation, microwave energy may be applied to induce current in the electrically conductive member and heat the crosslinkable material of the implant.

Thermally Crosslinkable Materials

Any appropriate thermally crosslinkable material may be used. Examples of thermally crosslinkable materials include thermally crosslinkable proteins such as albumin, collagen and fibrin. Other thermally crosslinkable materials may include carbohydrates, as well as synthetic polymers (e.g., plastics such as thermosetable materials and thermopastics). In general, the thermally crosslinkable material is applied to the implant or directly to the tissue in a substantially uncrosslinked state, so that current applied by the electrically conductive structure of the implant can crosslink (or further crosslink) it.

As described above, the thermally crosslinkable material may be selected or modified so that the resistivity of the material may be higher than that of the tissue into which it is implanted. For example, the thermally crosslinkable material may have an initial electrical resisitvity of greater than about: 100 Ohms*cm, 200 Ohm*cm, 500 Ohm*cm, 10 Ohm*m, 20 Ohm*m, 50 Ohm*m, 100 Ohm*m, etc. (including any intermediate values).

The resistivity of the thermally crosslinkable material may be modified by crosslinking or by a change in temperature. For example, the resistivity may be increased or decreased by the application of electrical energy. The resistivity of some thermally crosslinkable materials increase as they polymerize, which may further enhance heating (and further crosslinking) of the crosslinkable material.

A thermally crosslinkable material may be applied to an implant in any appropriate way, and may be applied so that it contacts and/or covers the electrically conductive structure of the implant. For example, the material may be dipped, sprayed, painted, layered, etc. The thermally crosslinkable material typically coats the entire exposed surface of the electrically conductive structure of the implant. The thermally crosslinkable material may form a thick or thin layer on the implant. For example, the thermally crosslinkable material may be coated to an approximate average thickness of 10 μm, 20 μm, 50 μm, 75 μm, 100 μm, 150 μm, 200 μm, 500 μm, etc.

One class of thermally crosslinkable materials includes collagens. Collagen typically consists of globular units of the collagen sub-unit tropocollagen. Tropocollagen sub-units spontaneously arrange themselves under physiological conditions into staggered array structures that may be stabilized by numerous hydrogen and, covalent bonds. The physical and electrical properties of collagen may vary. In one variation, collagen material may be prepared by dissolving a predetermined amount of collagen material in water to from a solution, applying the material to the implant, and drying or freeze drying the material on the implant surfaces (e.g., the electrically conductive surfaces that are to contact the tissue). The collagen material may be a mixture of an insoluble collagen material and a soluble collagen material, in one variation having a weight ratio of about 1:3 to 3:1.

Another class of thermally crosslinkable materials includes the albumins. Albumins are proteins (including ovoalbumins, human albumins, serum albumins, transgenic albumins, etc.) that may be crosslinkable or coagulable by heat. Albumins may be prepared dry, brought into solution and coated or otherwise applied to the devices (e.g., the electrically conductive surfaces of the devices), and allowed to dry.

Other classes of thermally crosslinkable materials may include fibrinogens, keratins, elastins, hyaluronic acids, and myoglobins.

As mentioned previously, the thermally crosslinkable materials may be mixtures of materials, including any of the proteins or polymers described herein and other components, including buffers, salts, proteins, carbohydrates, or the like. Some components of the thermally crosslinkable materials may not, themselves be crosslinkable. In some variations, additional components are included as part of the thermally crosslinkable material in order to increase the electrical resistivity of the thermally crosslinkable material (e.g., polar, electrophiles, etc.). Other materials may also be included as part of the thermally crosslinkable material, including materials that encourage growth (e.g., growth factors), or that prevent infection or contamination (e.g., antimicrobials, antibacterials, antifungals, etc.).

As mentioned briefly above, the thermally crosslinkable material may be applied to the device (or to directly to the tissue) in a substantially uncrosslinked state. For example, in some variations the thermally crosslinkable material is substantially denatured. For example, albumen may be applied in a substantially globular form. In some variations, the thermally crosslinkable material is partially crosslinked (e.g., where the number of linked multimers, n, is between 1 and 10, 1 and 20, 1 and 50). In some variations, the thermally crosslinkable material is applied to the device in a substantially crosslinked state, so that activation of the electrically conductive material first denatures the material, allowing it to renature (e.g., upon cooling after electrical current is reduced or terminated).

FIGS. 2A-E and 4 illustrate different features of the implants described herein. For example, an implant may be configured as a disposable or single-use adhesive pad which can be activated by electric current. A thin conductive layer (e.g., a foil, mesh, fabric, etc.) may be covered by a layer of thermally crosslinkable material (e.g., albumin) on both sides. When an electric current is applied to the conductive layer, the current passes through the thermally crosslinkable material and into the subject's body. As described above, this causes a local temperature rise in the thermally crosslinkable material, polymerizing the material. Once the material is adequately polymerized, the current may be turned off.

The current applied to the implant may be any electrical current adequate to heat and thereby polymerize the thermally crosslinkable coating. The current may be applied continuously or variably. When the current (or voltage) applied is variable, it may have a frequency (e.g., 10 Hz, 100 Hz, etc.). In some variations, electrochemical erosion of the implant and gas formation may be avoided by applying a current comprising an alternating current having a frequency above 100 kHz. In some variations a relatively low power may be applied over time (either continuously or in pulses) to crosslink the thermally crosslinkable material and/or erode the electrically conductive structure. For example, the voltage applied may be 800V, 700V, 600V, 500V, 400V, 300V, etc. In some variations, bipolar waveforms (e.g., ±Voltage) is applied.

In some variations, the electrical energy applied (e.g., current or voltage) is matched to the implant or to the thermally crosslinkable material(s) of the implant. Electrical energy may be applied to the implant to raise the temperature of the thermally crosslinkinkable material enough to crosslink the material to the tissue, but not enough to damage the thermally crosslinkable material or the surrounding tissue. For example, sufficient electrical energy may be applied to raise the temperature of the thermally crosslinkable material within a temperature range (e.g., less than 100° C., between 50° C. and 100° C., etc.). The relationship between applied energy and temperature may be calculated (e.g., see equations 1 and 2) for the implant, or may be determined experimentally. In some variations, the implant may include a temperature sensor or may provided feedback of the temperature of the implant and/or the surrounding tissue.

As described above, a rise in the temperature of the thermally crosslinkable material of the implant may be local to the material while avoiding excessive heating of adjacent tissues by using a thermally crosslinkable material having a resistivity that is higher than that of the adjacent tissue. For example, when collagen is used as a thermally crosslinkable material, it may have an ion concentration that is significantly less than that of the tissue, including other collagen present in the tissue or extracellular space. Since the current flowing from the electrically conductive region must first flow through the high-resistivity region of the coating, the Joule heat deposition preferentially heats the coating rather than the lower-resistivity tissue. Thus, resistivity can be optimized to control curing of the thermally crosslinkable material while minimizing the thermal damage to the tissue.

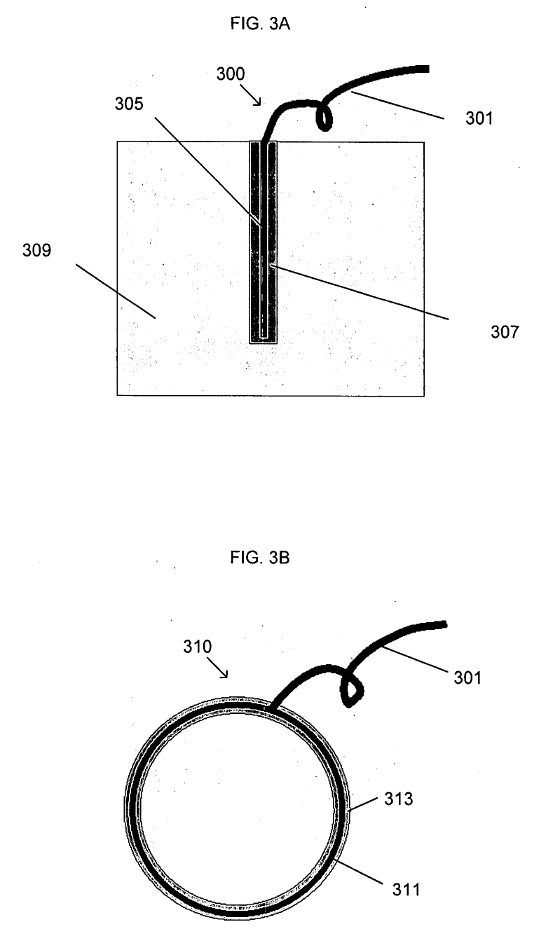

Tissue welding may be achieved using any of the devices described herein. An electrically activated adhesive film (implant) allows one-shot uniform welding of the tissue on wounds. An electrically adhesive welding device can have different shapes, including a planar sheet-like shape as shown in cross-section in FIG. 3A, and a pipe-like stents shape for tracheal connection and intra-luminal or extra-luminal anastomosis (e.g., reconnection of the cut blood vessel), as shown in cross-section in FIG. 3B. In FIG. 3A, the implant 300 is configured inserted into a cut or tear in the tissue 309. The implant 300 includes an electrically conductive structure (surface 305) and a thermally conductive material 307 that surrounds this exposed electrically conductive structure. A connector (wire 301) is releasably or frangibly connected to the implant, and can be connected to an electrical generator. A reference or ground electrode (e.g., a ground plate, not shown) may also be used. In this example, the sides of the tissue are placed immediately adjacent to the implant 300 so that when it is activated it will glue the tissue in both sides of the tissue tear 309. For example, pressure may be applied to secure the sides of the tissue until activation of the implant. Alternatively, additional thermally crosslinkable material may be placed into the tissue tear to further surround or coat the implant.

FIG. 3B is another example of an implant 310 having an electrically conductive surface 311 that is completely coated or surrounded by a thermally crosslinkable material 313, as shown. The implant 310 is a tubular implant having a circular cross-section. Although in FIG. 3B both sides of the implant (both sides of the electrically conductive structure 311) are coated with a crosslinkable material 313, in some variations, only a single side is coated with the thermally crosslinkable material. The uncoated side may be insulated or open to a high-impedance pathway (e.g., through air).

FIG. 4 is an example of an implant configured as a stent, as described above. Tissue welding using an electrically conductive structure 405, 405′ coated with a thermally crosslinkable material 407 may be used to prevent migration of a stent 401 in an aortic aneurism. The stent 401 shown in FIG. 4 has a metal or polymeric body 403, and multiple electrically conductive regions 405, 405′ that are connectable to a power supply via a connector (not shown). The electrically conductive regions 405, 405′ in FIG. 4 are configured as rings that are embedded or otherwise secured to the stent body 403. Portions of the electrically conductive structure (rings 405, 405′) are un-insulated and face outwards towards the tissue (when inserted into a subject), but are coated with a thermally crosslinkable material 407 for activation and electrical tissue welding. In this example, tissue may adhere to these pads (e.g., electrode regions 405, 405′) on the stent, preventing migration of the stent from the aneurism site. For example, a stent such as that shown in FIG. 4 may be inserted into a subject at an appropriate site within the body, expanded to fit the side, and activated by applying current to the electrically conductive regions to thermally crosslink the thermally crosslinkable material so that they are secured into position.

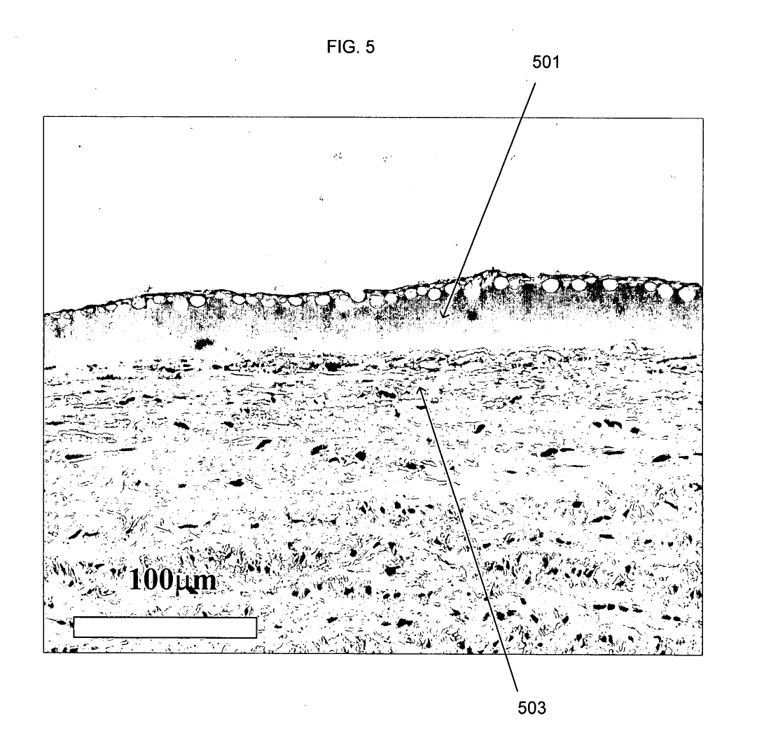

FIG. 5 is an example of a proof-of-concept experiment in which a 2 mm by 5 mm patch implant has been welding to the inner region of a porcine aorta. In FIG. 5 an implant including an electrically conductive surface and a coating of albumin 501 have been adhered to the endothelium 503 of the aorta. In this example, albumin was applied on the surface of gold coated captan foil 15 micrometers in thickness. The gold coating is electrically conductive and may also be degradable during activation. The thin layer of gold coating is typically approximately 10-50 nm in thickness. During activation of the device (the albumin coated gold foil), the gold electrically conductive layer degrades, and therefore there is no electrode visible on the top of the albumin layer.

As mentioned, albumin is the thermally crosslinkable material in the example shown in FIG. 5. The albumen used in this example was Grade V 98% crystallized salt-free desiccated albumin (SIGMA production, Lot 032K7029), and was dissolved in distilled de-ionized water to a concentration of 30% by weight, and then applied to the metallized 2×5 mm foil and vaporized to form a layer 100-300 micrometers in thickness.

The device was then applied to the inner surface of the artery. Electrical energy was then applied. In particular, a sinusoidal wave of 100 kHz, with 600V peak to peak, was applied to the electrically conductive structure (the metal coating) for 2-5 seconds. During the 2-5 seconds of activation, the metal was etched and the thermally crosslinkable albumin adhered to the tissue. Afterwards, the sample was examined by histological fixation.

Measurements of the tear stress for implants as described herein were performed using a copper foil (2×5 mm in size and 30 micrometer in thickness) attached to a 1 mm plastic substrate, similar to the gold foil example just descried. Force was applied to the substrate and the tissue.

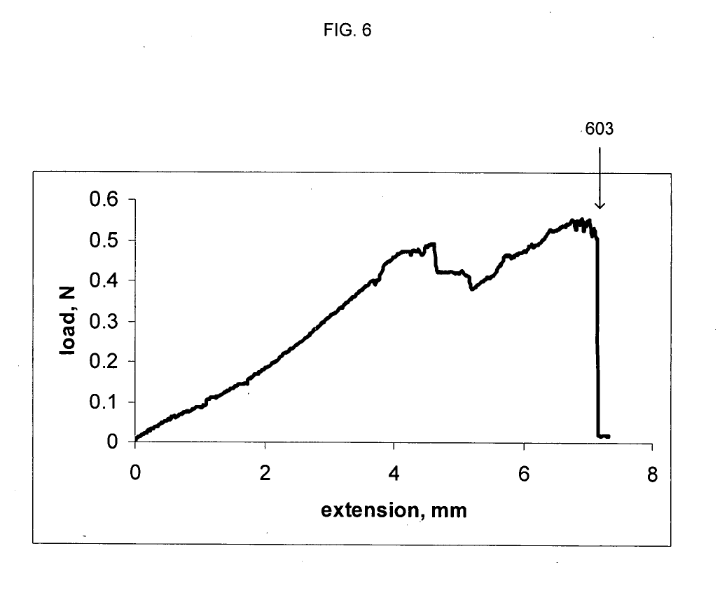

The electrical tissue welding performed by the methods, devices and systems described herein may form a strong bond to the tissue. An example of the strength of the tissue weld that may be formed is shown in FIG. 6, which illustrates a loading curve for an implant similar to the implant illustrated in FIG. 5, which includes a coating of albumin. As FIG. 6 illustrates, the tissue weld is formed using a 2 mm by 5 mm patch implant can withstand loading of up to just over half (0.5) a Newton (N), before failure 603. As the load increases, the welded tissue extends virtually linearly until failure. As the load increases, the welded tissue extends virtually linearly during elastic (reversible) deformation and begins rupturing during plastic (non-reversible) deformation until failure.

Any of the devices described herein may also be used as part of a system or method for adhering tissue to an implant or to other tissue. Methods of attaching an implant to a biological tissue may include any of the steps already described above. For example, an implant (e.g., an implant having an electrically conductive region coated with a thermally crosslinkable material) is inserted into the body (e.g., into the tissue of the body) at or near the region where it is desirable to cause tissue adhesion. When the tissue adhesion method is being used to cause adhesion of a device or implant (e.g., a stent, pacemaker, etc.), the device may be inserted into the tissue and placed adjacent to (e.g., contacting) the tissue to which the implant will be welded. Once the implant is in position, electrical energy is applied to the electrically conductive region to activate the thermally crosslinkable material and cause adhesion of the tissue. In some variations, the tissue maybe manipulated for optimal placement before activation of the implant to polymerize the thermally crosslinkable material.

After the implant has been sufficiently polymerized to cause adhesion, the implant may be disconnected from the power supply. As described above, this may mean disconnecting a plug, breaking a frangible connection, cutting the connection, or otherwise decoupling the connection from the implant.

As mentioned above, the device and/or techniques described herein may also be adapted to limit or stop bleeding. In particular, bleeding which may arise when cutting vascularized tissues. For example, when cutting an organ such as the liver, bleeding (often severe bleeding) may be problematic. Typically, bleeding is limited by using deep coagulation. Unfortunately, this method may damage a significant part of the tissue due to the high level of energy (e.g., heat) used. The methods and systems described herein may be used to treat bleeding by polymerizing a thermally crosslinkable material (e.g., albumin or fibrin etc.). Activation of the thermally crosslinkable material (e.g., thermal activation by applying electrical energy) will seal the bleeding blood vessels. As described above, an implant comprising an electrically conductive region in electrical communication with a thermally crosslinkable material may be used to seal blood vessels. Other examples or applications of the methods, devices and systems described herein may be apparent to one of skill in the art.

As described above, the methods, devices and system descried herein may be used in any appropriate application, including wound closure and healing, and the like. For example, the devices and methods described herein may be used to repair an annulus, including a spinal annulus, during an intervertebral disc repair or surgery.

The intervertebral disc is typically located between each vertebra, and can be described as biological shock absorber, helping to absorb pressure and preventing the spinal bones from rubbing against each other. Each disc has a strong outer ring of fibers called the annulus, and includes a soft, jelly-like center called the nucleus pulposus. The annulus helps keep the disc's center intact. In order to access the disc nucleus, e.g., during surgical procedures such as replacements and reductions, it is often necessary to make a cut or incision through the annulus region. However, the annulus is difficult to repair, as it is not highly vascularized and does not respond to traditional electrosurgical techniques. For example, it is often undesirable to suture this region because of the potential irritation to adjacent nerves.

It may therefore be desirable, for example, to use the methods and devices for tissue welding described herein to repair the disc annulus. In one variation, a cut (or tear) in the disc annulus may be repaired apply a thermally crosslinkable material (e.g., albumin) to the cut or tear, and applying electrical to the crosslinkable material by an implant, as described herein. For example, an implant may be an erodible implant (e.g., a metal foil or mesh) that erodes during the stimulation required to thermally crosslink the thermally crosslinkable material. In one variation, an implant having a coating of a thermally crosslinkable material is applied into the cut or tear (additional thermally crosslinkable material may be added). The implant may conform to the sides of the cut or tear, so that when the implant is activated, the thermally crosslinkable material joins the sides of the cut or tear, effectively welding the two together. If an erodible conductive material is used as part of the implant, the welded wound will not include the embedded electrically conductive region after the tissue has been welded.

The above detailed description is provided to illustrate exemplary embodiments and is not intended to be limiting. For example, any of the features of an embodiment may be combined with some or all of the features of other embodiments. It will be apparent to those skilled in the art that numerous modifications and variations within the scope of the present invention are possible. Throughout this description, particular examples have been discussed, including descriptions of how these examples may address certain disadvantages in related art. However, this discussion is not meant to restrict the various examples to methods and/or systems that actually address or solve the disadvantages. Accordingly, the present invention is defined by the appended claims and should not be limited by the description herein.

What is claimed is:

1. An implant configured to adhere to a biological tissue when activated by electrical energy, the implant comprising:
an electrically conductive structure having at least one uninsulated tissue-facing surface;
a connector releasably connected to the electrically conductive structure; and
a thermally crosslinkable coating, wherein the thermally crosslinkable coating covers the uninsulated tissue-facing surface of the electrically conductive structure.

2. The implant of claim 1, wherein the uninsulated tissue-facing surface of the electrically conductive structure comprises a pad.

3. The implant of claim 1, wherein the electrically conductive structure comprises a frame.

4. The implant of claim 1, wherein the uninsulated tissue-facing surface of the electrically conductive structure comprises an external region of a stent.

5. The implant of claim 1, wherein the electrically conductive structure comprises a foil.

6. The implant of claim 1, wherein the electrically conductive structure comprises a mesh.

7. The implant of claim 1, wherein the electrically conductive structure comprises a metal selected from the group consisting of: titanium, gold, nickel, implant-grade stainless steel, cobalt alloys, or platinum.

8. The implant of claim 1, wherein the electrically conductive structure comprises an electrically conductive material configured to degrade during activation to thermally crosslink the thermally crosslinkable material.

9. The implant of claim 1, wherein the electrically conductive structure comprises a bioabsorbable material.

10. The implant of claim 1, wherein the connector is releasably connected by a frangible connection.

11. The implant of claim 1, wherein the connector is releasably connected by an electrically erodible connection.

12. The implant of claim 1, wherein the connector is a plug.

13. The implant of claim 1, wherein the thermally crosslinkable coating has a resistivity higher than biological tissue.

14. The implant of claim 1, wherein the thermally crosslinkable coating has a resistivity higher than 100 Ohm*cm.

15. The implant of claim 1, wherein the resistivity of the thermally crosslinkable coating increases during thermal crosslinking.

16. The implant of claim 1, wherein the thermally crosslinkable coating comprises albumin.

17. The implant of claim 1, wherein the thermally crosslinkable coating is selected from the group consisting of: collagen, fibrin, or polysaccharide.

18. The implant of claim 1, wherein the thermally crosslinkable coating is greater than 10 μm thick.

19. The implant of claim 1, wherein the thermally crosslinkable coating comprise a substantially uncrosslinked material.

20. The implant of claim 1, further comprising an insulating layer over a region of the electrically conductive structure.

21. An electrically activated adhesive implant configured to adhere to a biological tissue when activated by electrical energy, the implant comprising:
an electrically conductive structure having an uninsulated tissue-facing region, where the electrically conductive structure is configured to connect to a power supply via a frangible connector,
wherein the uninsulated tissue-facing region of the electrically conductive structure is coated with a thermally crosslinkable coating, the thermally crosslinkable coating having a resistivity higher than 100 Ohms*cm.

22. An electrically activated adhesive implant configured to adhere to a biological tissue when activated by electrical energy, the implant comprising:
an electrically conductive structure having an uninsulated tissue-facing region;
a connector configured to connect the electrically conductive structure to a power supply,
wherein the uninsulated tissue-facing region of the electrically conductive structure is coated with a thermally crosslinkable coating comprising albumin.

23. A method of attaching an implant to a biological tissue, comprising:
inserting an implant into the tissue, wherein the implant comprises:
an electrically conductive structure having an uninsulated tissue-facing region;
configured to connect to a power supply, wherein the uninsulated tissue-facing region is in electrical contact with a thermally crosslinkable material;
applying current through the thermally crosslinkable material from the uninsulated tissue-facing region to activate the thermally crosslinkable coating.

24. The method of claim 23, further comprising:
connecting the electrically conductive structure to a power supply.

25. The method of claim 23, wherein the thermally crosslinkable material comprises albumin.

26. A method of welding a biological tissue, comprising:
placing an implant adjacent to the tissue, wherein the implant comprises:
an electrically conductive structure; and
a thermally crosslinkable coating covering at least a portion of the electrically conductive structure,
so that the thermally crosslinkable coating is between the electrically conductive structure and the tissue;
applying electrical energy to the electrically conductive structure of the implant to at least partially crosslink the thermally crosslinkable coating of the implant with the tissue.

27. The method of claim 26, further comprising applying electrical energy to the electrically conductive structure of the implant at least until the electrically conductive structure substantially erodes.

28. A method of welding a biological tissue, comprising:
making an electrical connection between an implant and a power supply, wherein the implant comprises:
an electrically conductive structure; and
a thermally crosslinkable coating covering at least a portion of the electrically conductive structure;
placing the thermally crosslinkable coating of the implant against to the tissue; and
eroding the electrically conductive structure and crosslinking the thermally crosslinkable coating by applying electrical energy to the electrically conductive structure of the implant.
